(19) United States
(12) Patent Application Publication
(10) Pub. No.: US 2003/0077617 A1
(43) Pub. Date: Apr. 24, 2003

(54) Method for diagnosis of a disease by using multiple SNP (single nucleotide polymorphism) variations and clinical data

(76) Inventors: Myungho Kim, East Brunswick, NJ (US); Gene Kim, East Brunswick, NJ (US)

Correspondence Address: MYUNG HO KIM, 93 B TAYLER AVE., EAST BRUNSWICK, NJ, 08816, US

(21) Appl. No.: 10/128,377

(22) Filed: Apr. 24, 2002

(30) Foreign Application Priority Data
Oct. 24, 2001 (KR) 10 2001-0064130

(51) Int. Cl. C12Q 1/68; G06F 19/00; G01N 33/48; G01N 33/50

(52) U.S. Cl. 435/6; 702/20

(57) ABSTRACT

A method comprises the step of representing a pair of genotypes at an SNP location, and/or clinical data, as a single number or a vector. Moreover, the method further comprises the step of applying a support vector machine to at least two of such vectors so as to optimally classify the vectors into one of the at least two subgroups. There is a particular application as a method for diagnosing a disease by representing a person or an organism as the above-type of vectors and then obtaining a cutoff hypersurface by applying a support vector machine to the vectors, wherein the cutoff surface serves to separate and classify the vectors into the at least two subgroups, the first with a disease and the second without.

This application is related to and claims priority from Korean Patent Application No. 10-2001-0064130, filed Oct. 24, 2001, which is incorporated herein by reference in its entirety.

### BACKGROUND OF THE INVENTION

1. Technical Field

The present invention relates to a method, comprising the step of representing a pair of genotypes at an SNP location, and/or clinical data, as a single number or a vector. Moreover, the present invention further comprises the step of applying a support vector machine to at least two of such vectors so as to optimally classify the vectors into one of the at least two subgroups.

The present invention has particular application as a method for diagnosing a disease by representing a person or an organism as the above-type of vectors and then obtaining a cutoff hypersurface by applying a support vector machine to the vectors, wherein the cutoff surface serves to separate and classify the vectors into the at least two subgroups, the first with a disease and the second without.

2. Description of the Related Art

Since the completeness of human genome sequence was announced, there has been a lot of excitement in the hope of deciphering the sequences and discovering new drugs for diseases. However, the obtained results did not meet the expectations because researchers were not successful in developing a new method suitable for the current situation, and there is no standard method to analyze the great amount of genome data. As a result, scientists have been slowed down in taking advantage of the complete human sequence.

So the new concepts and novel approach for analyzing not only the genetic data but also existing clinical data are urgently needed. More precisely, there is a need to develop a new method and concept of dealing with many variables simultaneously, instead of looking at a variable one by one.

Along this line, the present invention introduces a completely new concept in the emerging area of bioinformatics by applying machine-learning methods to genome and clinical data for appropriate diagnosis and analysis.

### SUMMARY OF THE INVENTION

The present invention opens up a new horizon to medical diagnosis and analysis of biological data, and contributes to enhance health care for persons. Traditionally, doctors set a normal range of blood pressure based on data obtained from a large number of people. If a patient is excluded from the range, the doctors tried to “set it right.” Over the years, people have observed the fact that some healthy people are not in the “normal range.” This fact implies that there are other factors than blood pressure that “cooperate” with the blood pressure factor to keep a person's health in balance. This makes us develop a new concept of analyzing multiple variables (contributing factors) simultaneously, not individually.

We start with two concepts.

1. In order to classify objects we are interested in, we need to find a new way of representing the objects into numbers.

2. To get a criterion (cutoff) used to divide a group, a knowledge-based method is needed.

Along the concepts above, we represent a group of objects into vectors. Then we label them and separate the group into two subgroups. From the division, we obtain a cutoff/criterion distinguishing one subgroup from the other subgroup. The cutoff will be used to determine, to which group, a new vector representation of an object belongs to.

### BRIEF DESCRIPTION OF THE DRAWINGS

The aforementioned aspects and other features of the invention will be explained in the following description, taken in conjunction with the accompanying drawings wherein:

FIG. 1 is a drawing of an embodiment of the present invention;

FIG. 2 is a drawing illustrating another embodiment of the present invention;

FIG. 3 is a drawing illustrating another embodiment of the present invention;

FIG. 4 is a drawing illustrating another embodiment of the present invention;

FIG. 5 is a drawing illustrating another embodiment of the present invention;

FIG. 6 is a drawing illustrating another embodiment of the present invention;

FIG. 7 is a drawing illustrating another embodiment of the present invention;

FIG. 8 is a drawing illustrating another embodiment of the present invention;

FIG. 9 is a drawing illustrating another embodiment of the present invention;

FIG. 10 is a drawing illustrating another embodiment of the present invention;

FIG. 11 is a drawing illustrating another embodiment of the present invention;

FIG. 12 is a drawing illustrating another embodiment of the present invention;

FIG. 13 is a drawing illustrating another embodiment of the present invention;

FIG. 14 is a drawing illustrating another embodiment of the present invention;

FIG. 15 is a drawing illustrating another embodiment of the present invention;

FIG. 16 is a drawing illustrating another embodiment of the present invention;

FIG. 17 is a drawing illustrating another embodiment of the present invention;

FIG. 18 is a drawing illustrating another embodiment of the present invention;

FIG. 19 is a drawing illustrating another embodiment of the present invention;

FIG. 20 is a drawing illustrating another embodiment of the present invention;

FIG. 21 is a drawing illustrating another embodiment of the present invention;

FIG. 22 is a drawing illustrating another embodiment of the present invention;

FIG. 23 is a drawing illustrating another embodiment of the present invention;

FIG. 24 is a drawing illustrating another embodiment of the present invention;

FIG. 25 is a drawing illustrating another embodiment of the present invention;

FIG. 26 is a drawing illustrating another embodiment of the present invention;

FIG. 27 is a drawing illustrating another embodiment of the present invention;

FIG. 28 is a drawing illustrating another embodiment of the present invention;

FIG. 29 is a drawing illustrating another embodiment of the present invention;

FIG. 30 is a drawing illustrating another embodiment of the present invention;

FIG. 31 is a drawing illustrating another embodiment of the present invention;

FIG. 32 is a drawing illustrating another embodiment of the present invention;

FIG. 33 is a drawing illustrating another embodiment of the present invention;

FIG. 34 is a drawing illustrating another embodiment of the present invention;

FIG. 35 is a drawing illustrating another embodiment of the present invention;

FIG. 36 is a drawing illustrating another embodiment of the present invention;

FIG. 37 is a drawing illustrating another embodiment of the present invention;

FIG. 38 is a drawing illustrating another embodiment of the present invention; and

FIG. 39 is a drawing illustrating another embodiment of the present invention;.

### DETAILED DESCRIPTION

As preliminary matter, the present invention is related to a paper authored by the inventors of the present invention, “Application of Support Vector Machine to detect an association between a disease or trait and multiple SNP variations,” which is incorporated herein in its entirety.

The present invention will be described in detail, with reference to the accompanying drawings.

Present invention is based on a new concept and it integrates with learning methods with SNP and/or clinical data. By way of background, the term, “numericalization” means representing some objects or properties of objects into a number or a vector. SNP is the short for single nucleotide polymorphism. The characters “A” and “B” will refer to some groups, which will vary depending on the context.

For example, before each concept was discovered, there were not concepts of height, weight, alcohol concentration in blood, speed limit, cholesterol level, and etc. But to measure and set some criterion for any objects people are dealing with, new ways of numericalization of certain properties were defined, whenever required. Along this line, we define a new way of numericalization of clinical data and/or SNP data and of classification into several groups, depending on what we want to analyze.

Given an SNP location, there are, in general, three types of genotypes such as ww, wm and mm (of course, in case more than three types, then we may add types such as m2m etc.). As is known, there are pairs of chromosomes and we have always a pair of genotypes. Here, w means wild genotype while m does mutation genotype. Wild type is found in the majority of people (or organisms) and mutation is not in the minority of people. Then we can do numericalization of ww, wm and mm. In other words, we assign different numbers or vectors to ww, wm and mm, as will be discussed further below with respect to the drawings.

For example, we may assign numbers 1, 2 and 3 to ww, wm and mm respectively. At the same SNP location, the numbers should be the same for all the persons (or organisms). But the numbers can vary as SNP location varies. From the description above, if we have N numbers of SNP locations, we have N numbers for each person (or a organism). By numbering the N numbers of SNP locations into SNP1, SNP2, . . . , SNPN, then, for each person(or a organism), those enumerated N numbers assigned to the N numbers of SNP locations form a vector in the N dimensional Euclidean space, as again, will be discussed further below with respect to the drawings.

For the second example, we may assign vectors (3, 0, 0), (0, 2, 1), (1, 0, 0.3) to ww, wm and mm respectively. Again as in the first example, at the same SNP location, the three vectors should be the same for all the persons (organisms). But the vectors can vary as SNP location varies. From the description above, if we have N numbers of SNP locations, we have N vectors for each person(or a organism). By numbering the N numbers of SNP locations into SNP1, SNP2 . . . , SNPN, then, for each person(or a organism), those enumerated N vectors assigned to the N numbers of SNP locations form a vector in the 3N dimensional Euclidean space.

As we explained in the two examples above, once we have numericalization of SNPs of persons(or organisms), we label each vector +1 or −1 accordingly. Suppose we have a group of persons(or organisms). Here are a few examples of labeling vectors. (1) Depending on whether the person (or the organism) represented by each vector has a specific disease or not, the vector is labeled by +1 or −1. (2) Given a disease, depending on whether the disease status of persons (or organisms) represented by each vector is at the stage, “A” or “B”, the vector is labeled by +1 or −1. (3) It is believed that each person has his/her own degree of radiation sensitivity due to genetic difference that may be distinguished by SNP data. Label a vector +1, if the person represented by the vector has the degree of radiation sensitivity, “A”, and −1 otherwise. In case there are more than two degrees, there is a way of solving the problem. (4) Given a drug, some people have some allergies against it while some do not. Label a vector +1 if the person represented by the vector has an adverse effect and −1 otherwise.

By applying classification methods such as support vector machine, neural network etc, we can find a cutoff to separate the set of +1 labeled vectors from the set of −1 labeled vectors with optimal errors. More precisely, the cutoff is determined by a hypersurface dividing the Euclidean space into two disjointed parts and will be used for determining whether an unlabeled vector representing a person(or a organism) should be labeled +1 or −1, accordingly the person has a specific disease or not. The same thing also works for (2), (3), and (4) above.

Suppose a cutoff hypersurface separates a Euclidean space into two parts, “A” and “B”. Also, suppose that “A” part contains more +1 labeled vectors than “B”, while “B” part do more −1 labeled vectors than “A”. We mean optimal errors by maximizing the rate of the set of +1 labeled vectors in “A” among the total number of labeled vectors of “A” and the rate of the set of −1 labeled vectors in “B” among the total number of labeled vectors of “B”. This is the optimal classification that we are referring to in the discussion below, as well (see, e.g., claims 8, and related drawing and description).

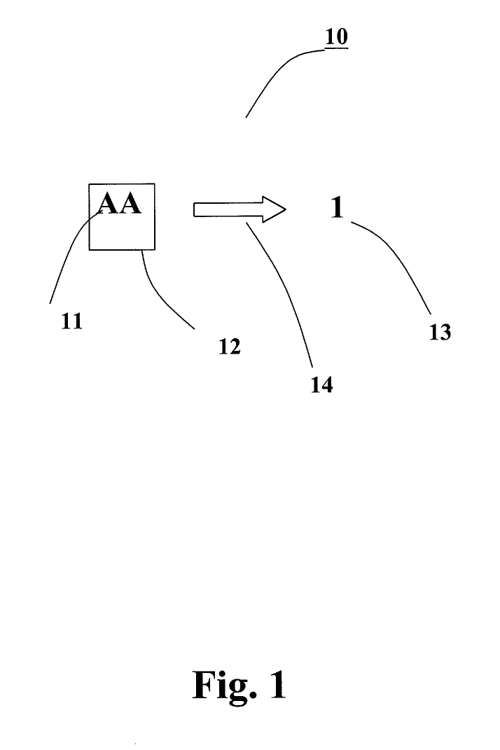

Turning to the drawings, FIG. 1 shows a drawing exemplifying the first embodiment according to the present invention. A method 10 comprises the step of representing (arrow 14) a pair of genotypes 11 (“AA”) at an SNP location 12 as a single number 1 (reference number 13). The phrase “single number” is meant to distinguish from numbers that are pair of numbers, such as two 1's or 11 being used to refer to wild-wild genotype. Thus, single number means a number such as 1, 2, 3, or 33 which stand for a single value and does not represent a combination of two numbers.

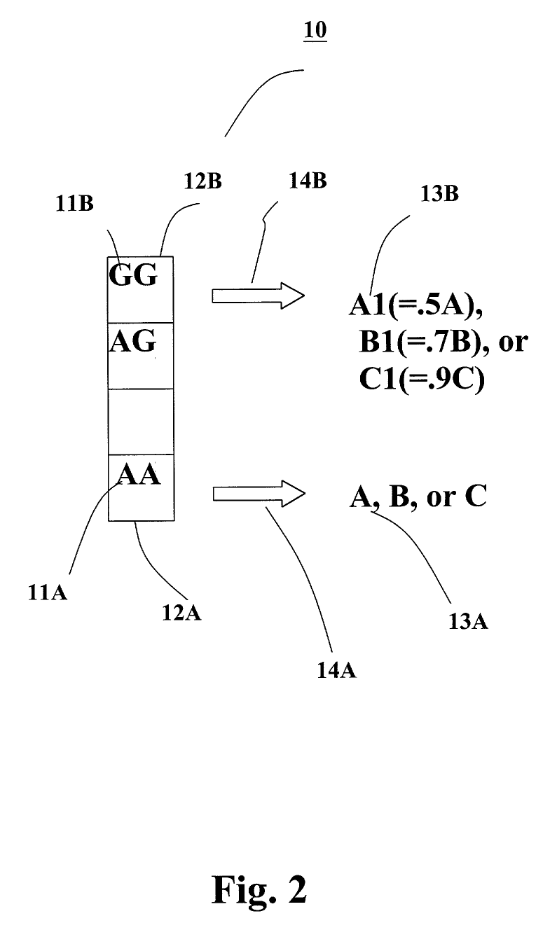

FIG. 2 shows a drawing exemplifying another embodiment according to the present invention, wherein the single number 13 of FIG. 1 comprises one of A, B, and C (reference number 13A), and wherein a relative value of the A,B, and C depend on the SNP location. Thus, at location 12B, for example, the relative value of A1, B1, and C1 differ from the relative value of A, B, and C at location 12A (with A1=0.5A, B1=0.7B, and C1=0.9C). For brevity sake, discussions relating to like reference numbered components of different drawing figures will not be repeated, but are incorporated herein.

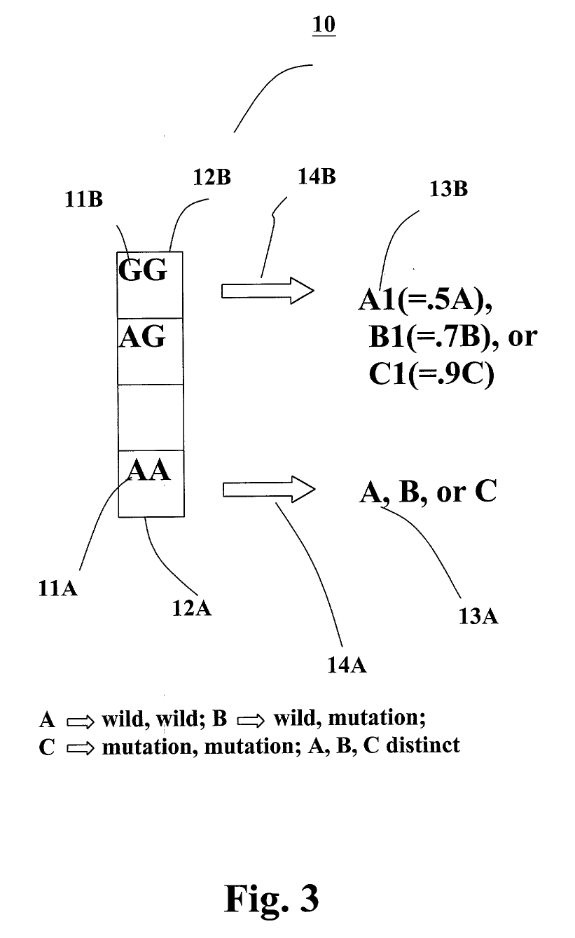

FIG. 3 shows a drawing exemplifying another embodiment according to the present invention. In a method according to the embodiment of FIG. 2, A corresponds to a pair of genotypes comprising a wild genotype and a wild genotype; B corresponds to a pair of genotypes comprising a wild genotype and a mutation genotype; and C corresponds to a pair of genotypes comprising a mutation genotype and a mutation genotype. Also, A, B, and C have distinct or different values. For example, A may have the value of 1, B may have the value of 2, and C may have the value of 3.

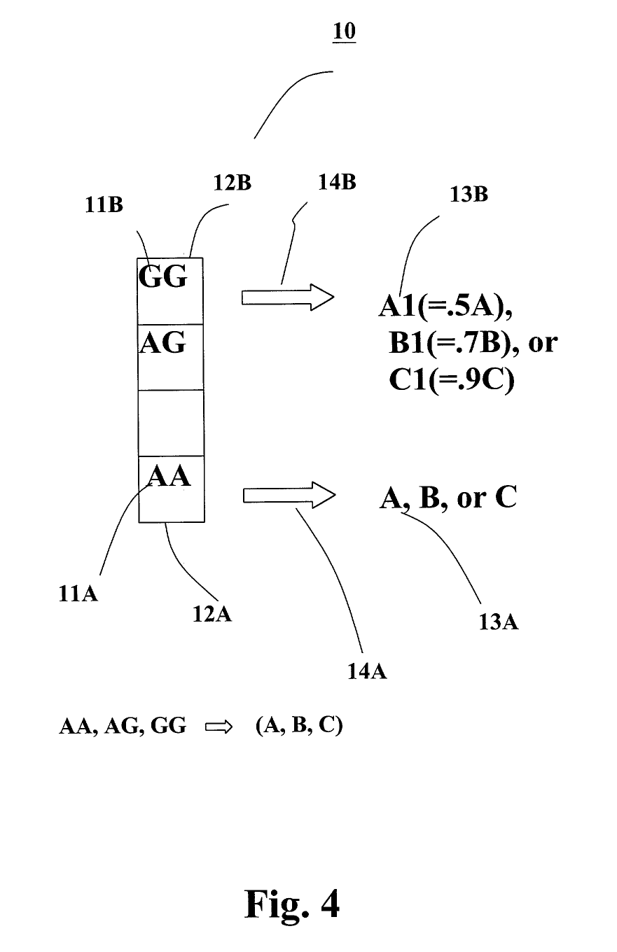

FIG. 4 shows a drawing exemplifying another embodiment according to the present invention. In the method according to the embodiment of FIG. 1, each one of a plurality of pairs of genotypes (11A, 11B, for example) at a respective one of a plurality of SNP locations (12A, 12B, for example) is represented as a respective one of a plurality of single numbers (A,B,C,A1,B1, or C1, for example), wherein the plurality of pairs of genotypes may be represented as a set of single numbers (A,B,C).

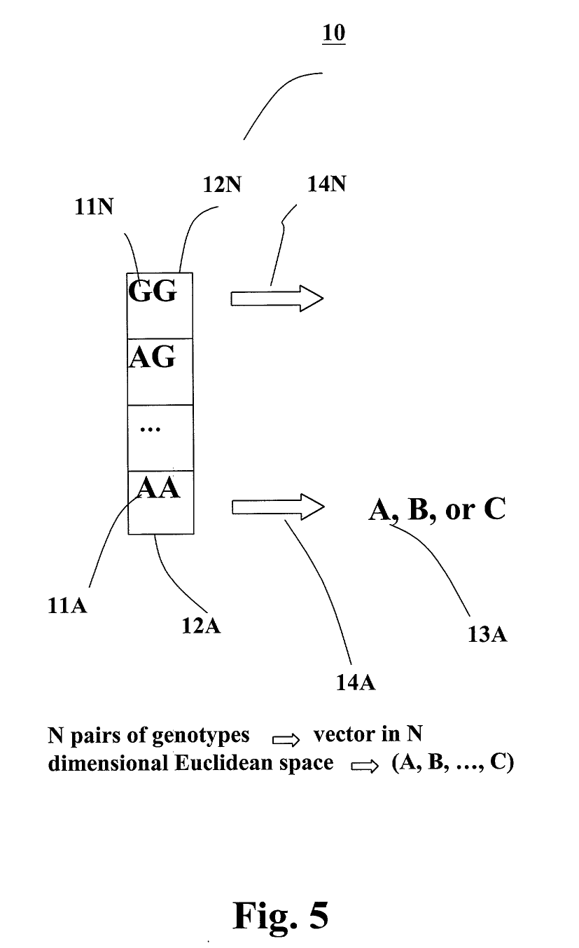

FIG. 5 shows a drawing exemplifying another embodiment according to the present invention. In the embodiment according to FIG. 4, N pairs of genotypes (11A . . . 11N) at a respective one of an N number of the plurality of SNP locations (12A . . . 12N) are represented as a vector in an N dimensional Euclidean space, wherein the vector comprises an N number of the plurality of single numbers, in a predetermined order, to be (A,B, . . . C).

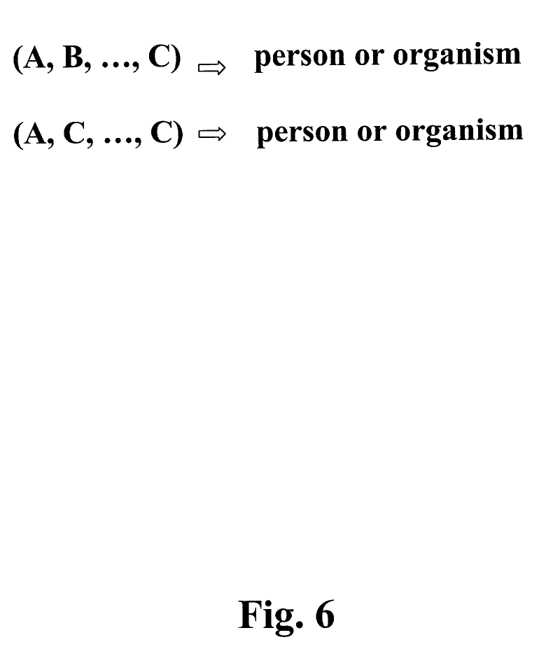

FIG. 6 shows a drawing exemplifying another embodiment according to the present invention. In a method according to FIG. 5, the vector (A,B, . . . C) corresponds to one of a person or an organism, and wherein the person or the organism belongs in one of at least two different classes of a person or an organism, wherein the at least two different classes differ by at least one different pair of genotype at an SNP location (here, for example, at the second location).

Thus, the present invention may be applied to persons, in diagnosing a disease for example, or to other organisms, such as a dog or perhaps another type of organism. Also, there of course may be more than two different classes and the classes may have more than one different pair of genotypes at an SNP location.

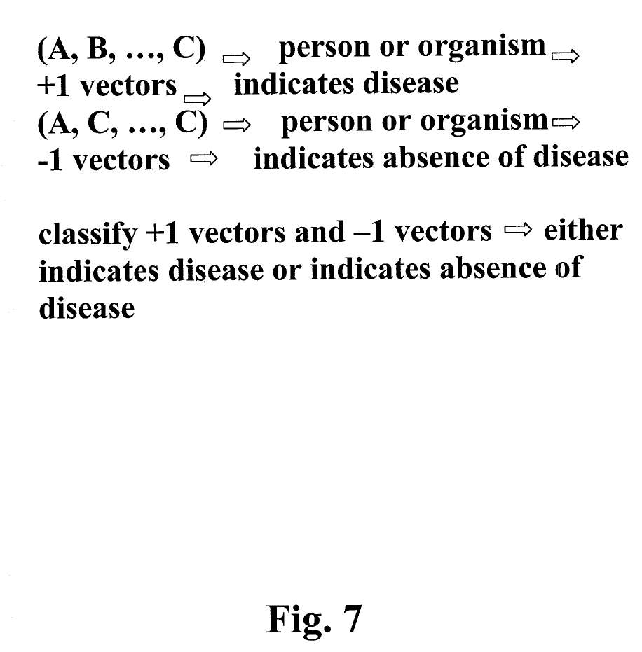

FIG. 7 shows a drawing exemplifying another embodiment according to the present invention. In a method according to FIG. 6, a person or an organism is represented as one of a labeled vector +1 and a labeled vector −1, wherein the labeled vector +1 indicates a disease and the labeled vector −1 indicates absence of the disease. Also, at least two of the labeled vectors corresponding to a respective one of a plurality of either a person or an organism are classified into either a group with at least two subgroups, wherein the first one of the at least two subgroups indicates the disease and the second one of the at least two subgroups indicates absence of the disease. Thus, in addition to what is shown in FIG. 7, there may, for example, be a vector (A, B, . . . B) that represents a person or an organism and that represent a state other than indicating disease and indicating absence of disease. One example of this might be a subgroup that indicates a latency for a disease (as opposed to full-blown form of the disease).

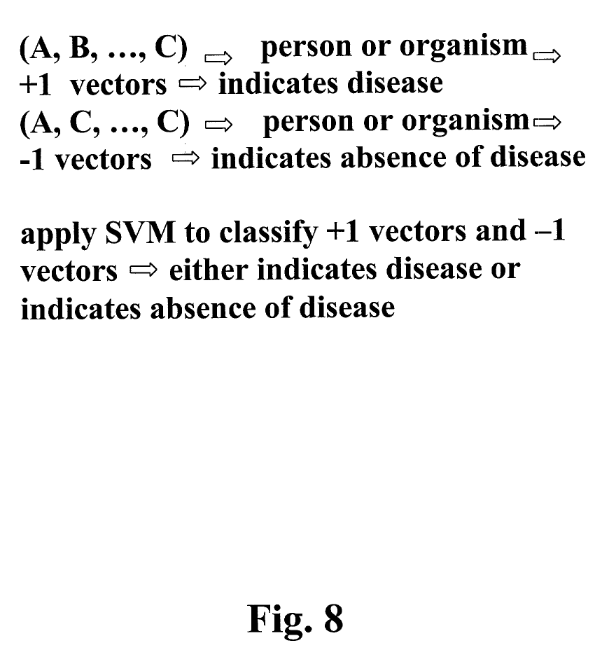

FIG. 8 shows a drawing exemplifying another embodiment according to the present invention. In a method according to FIG. 7, wherein the classifying step further comprises applying a support vector machine to the at least two labeled vectors so as to optimally classify the at least two labeled vectors into one of the at least two subgroups (please see above for discussion of optimization).

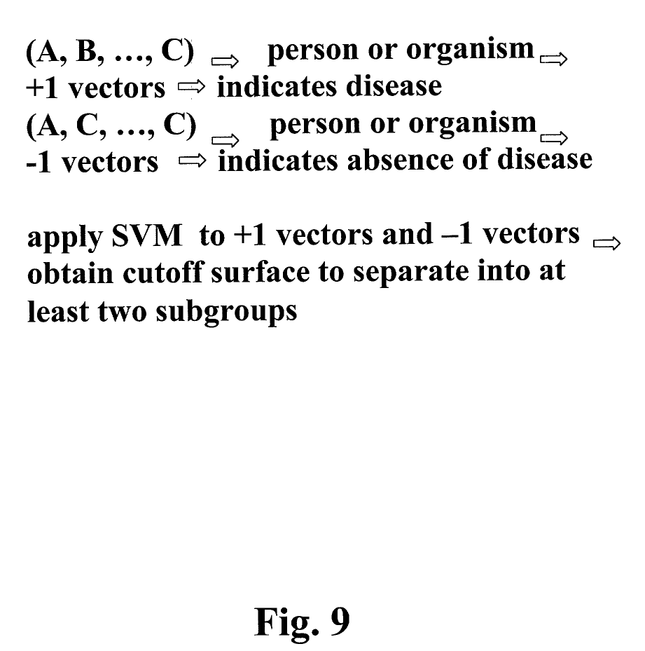

FIG. 9 shows a drawing exemplifying another embodiment according to the present invention. In a method according to FIG. 8, a cutoff hypersurface is obtained by applying the support vector machine to the at least two vectors, wherein the cutoff surface serves to separate and classify the at least two vectors into the at least two subgroups.

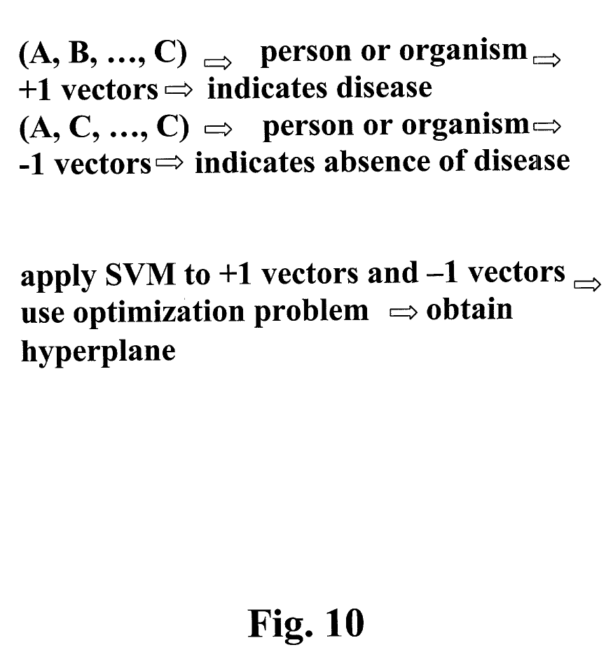

FIG. 10 shows a drawing exemplifying another embodiment according to the present invention. In a method according to FIG. 9, a hyperplane, which is a specific type of a cutoff surface, may be calculated by using an optimization problem comprising the following, wherein each yᵢ is +1 or −1 and xᵢ is a vector:

Maximize: W(α)=½Σ^{l}_{i,j=1}yᵢyⱼαᵢαⱼ(xᵢ·xⱼ)−Σ^{l}_{i,=1}αᵢ

Under the conditions Σ^{l}ᵢ₌₁αᵢyᵢ=0 and 0<=αᵢ<=C, i=1, 2 . . . l, wherein C is a given constant.

It may be worth noting that this hyperplane may be less accurate that the cutoff hypersurface in classification. In any event, by using either the hyperplane or the cutoff hypersurface, then one may be able to predict if a person has the genotype for the disease by numericalizing the SNP data (and the clinical data, for embodiment provided below) for the person.

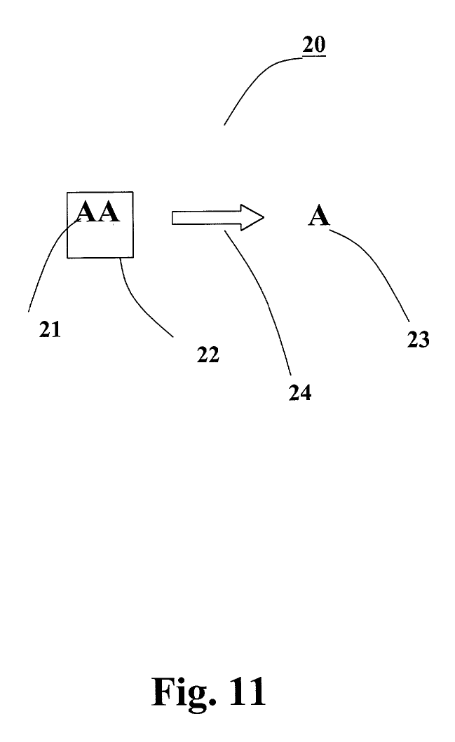

FIG. 11 shows a drawing exemplifying another embodiment according to the present invention. A method 20 comprises the step of representing (arrow 24) a pair of genotypes 21 (“AA”) at an SNP location 22 as a vector A (reference number 23).

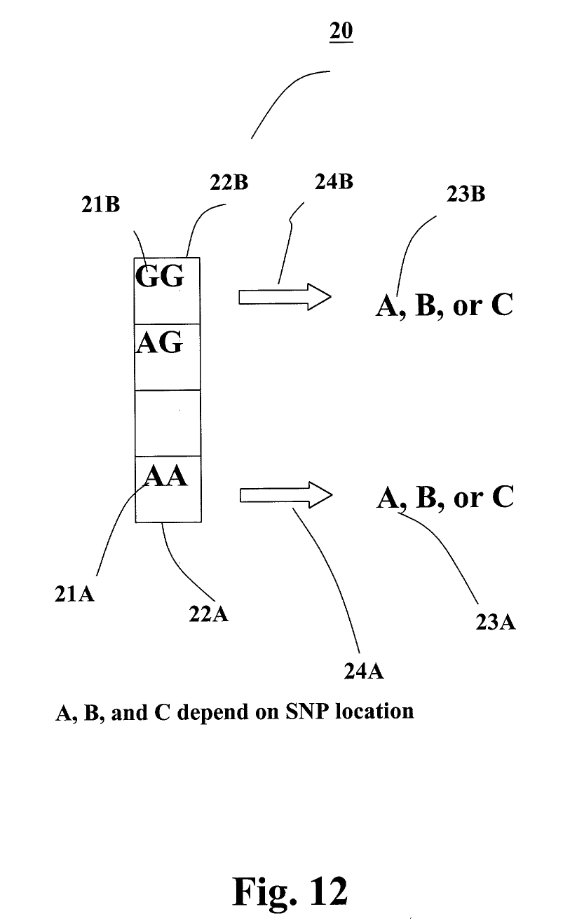

FIG. 12 shows a drawing exemplifying another embodiment according to the present invention, wherein the vector 23 of FIG. 11 comprises one of A, B, and C (reference number 13A), and wherein a relative value of the A,B, and C depend on the SNP location.

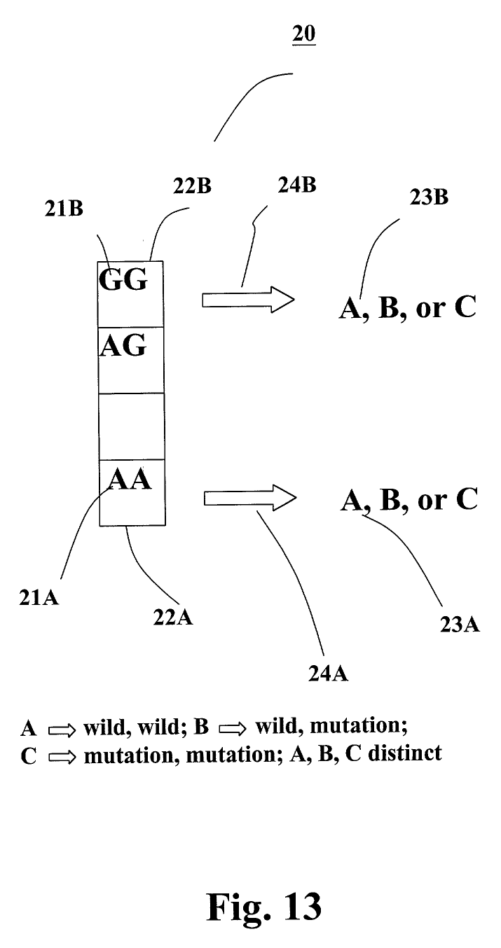

FIG. 13 shows a drawing exemplifying another embodiment according to the present invention. In a method according to the embodiment of FIG. 12, A corresponds to a pair of genotypes comprising a wild genotype and a wild genotype; B corresponds to a pair of genotypes comprising a wild genotype and a mutation genotype; and C corresponds to a pair of genotypes comprising a mutation genotype and a mutation genotype. Also, A, B, and C are distinct.

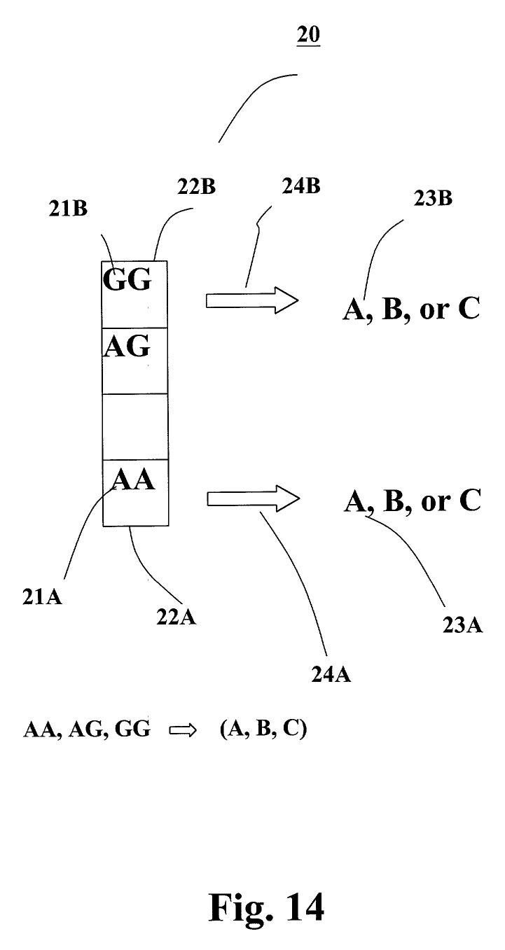

FIG. 14 shows a drawing exemplifying another embodiment according to the present invention. In the method according to the embodiment of FIG. 11, each one of a plurality of pairs of genotypes (21A, 21B, for example) at a respective one of a plurality of SNP locations (22A, 22B, for example) is represented as a respective one of a plurality of vectors (A,B, or C, for example), wherein the plurality of pairs of genotypes may be represented as a set of vectors (A,B,C).

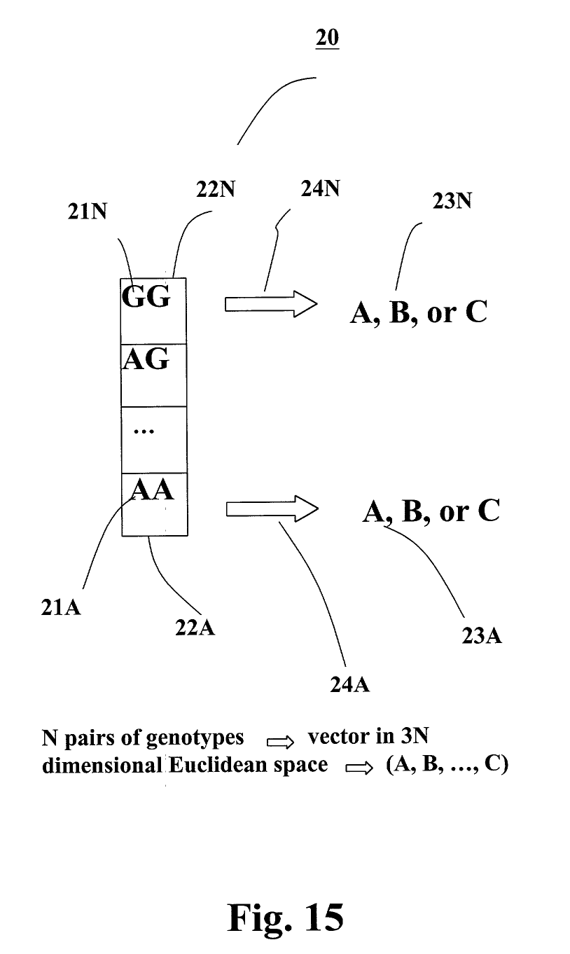

FIG. 15 shows a drawing exemplifying another embodiment according to the present invention. In the embodiment according to FIG. 14, N pairs of genotypes (11A . . . 11N) at a respective one of an N number of the plurality of SNP locations (12A . . . 12N) are represented as a vector in an 3N dimensional Euclidean space, wherein the vector comprises an N number of the plurality of single numbers, in a predetermined order, to be (A,B, . . . C).

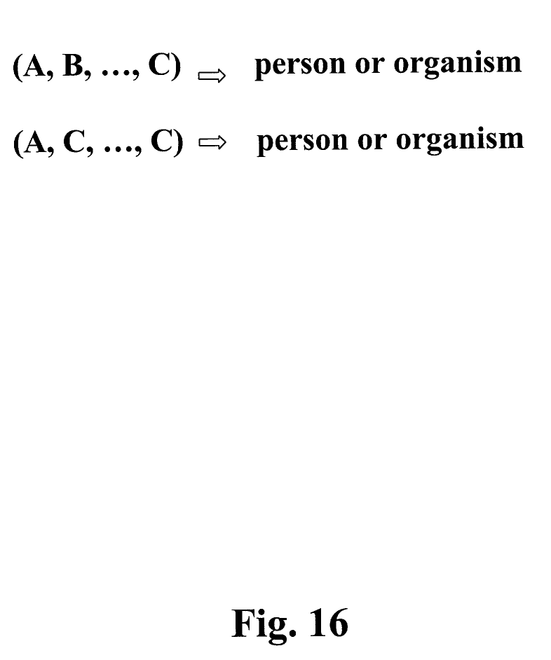

FIG. 16 shows a drawing exemplifying another embodiment according to the present invention. In a method according to FIG. 15, the vector (A,B, . . . C) corresponds to one of a person or an organism, and wherein the person or the organism belongs in one of at least two different classes of a person or an organism, wherein the at least two different classes differ by at least one different pair of genotype at an SNP location (here, for example, at the second location).

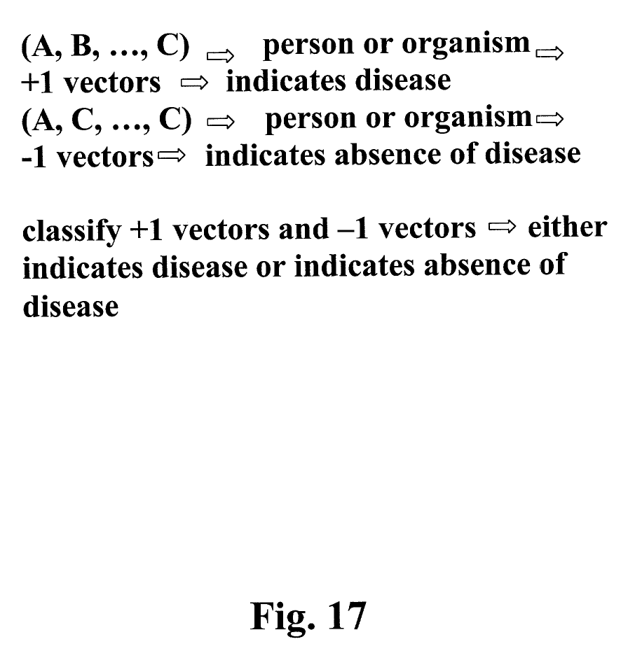

FIG. 17 shows a drawing exemplifying another embodiment according to the present invention. In a method according to FIG. 16, a person or an organism is represented as one of a labeled vector +1 and a labeled vector −1, wherein the labeled vector +1 indicates a disease and the labeled vector −1 indicates absence of the disease. Also, at least two of the labeled vectors corresponding to a respective one of a plurality of either a person or an organism are classified into either a group with at least two subgroups, wherein the first one of the at least two subgroups indicates the disease and the second one of the at least two subgroups indicates absence of the disease. Thus, in addition to what is shown in FIG. 17, there may, for example, be a vector (A, B, . . . B) that represents a person or an organism and that represent a state other than indicating disease and indicating absence of disease.

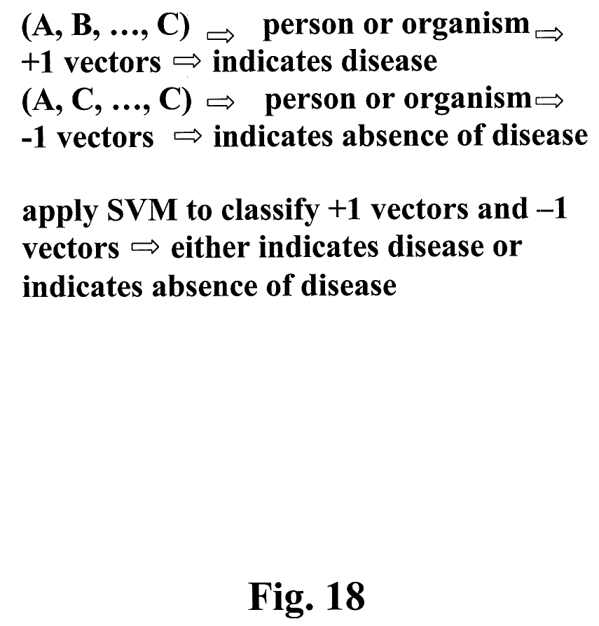

FIG. 18 shows a drawing exemplifying another embodiment according to the present invention. In a method according to FIG. 17, wherein the classifying step further comprises applying a support vector machine to the at least two labeled vectors so as to optimally classify the at least two labeled vectors into one of the at least two subgroups.

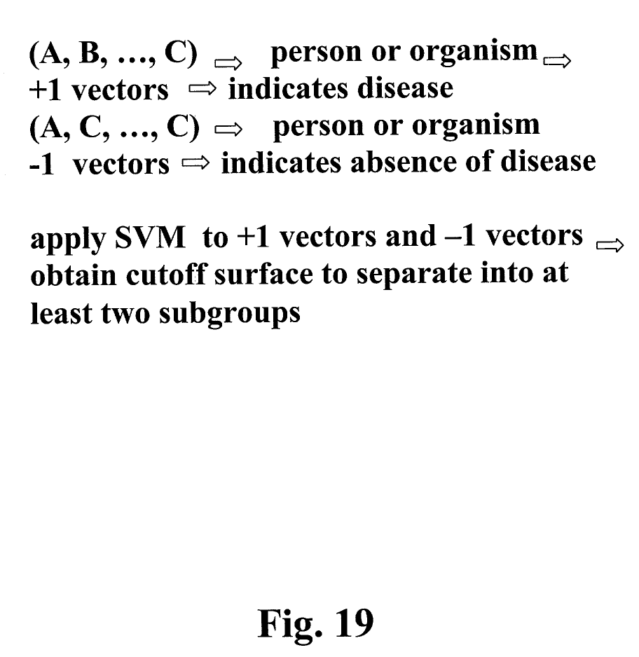

FIG. 19 shows a drawing exemplifying another embodiment according to the present invention. In a method according to FIG. 18, a cutoff hypersurface is obtained by applying the support vector machine to the at least two vectors, wherein the cutoff surface serves to separate and classify the at least two vectors into the at least two subgroups.

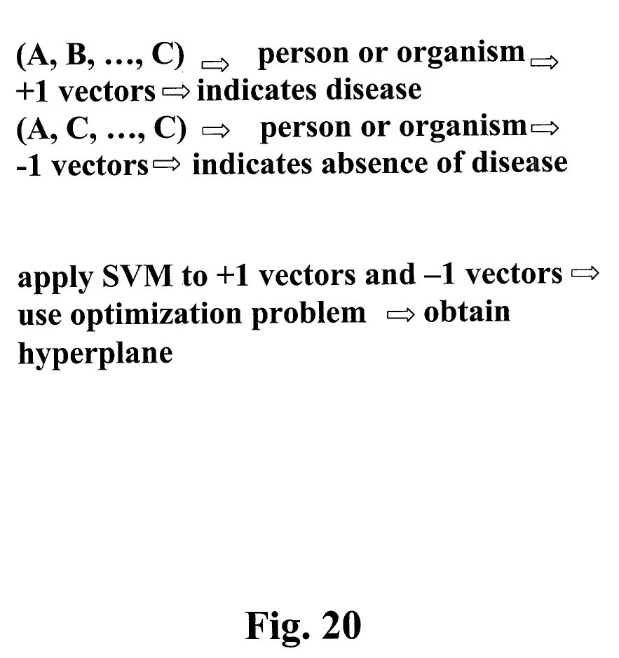

FIG. 20 shows a drawing exemplifying another embodiment according to the present invention. In a method according to FIG. 19, a hyperplane, which is a specific type of a cutoff surface, may be calculated by using an optimization problem comprising the following, wherein each yᵢ is +1 or −1 and xᵢ is a vector:

Maximize: W(α)=½Σ^{l}_{i,j=1}yᵢyⱼαᵢαⱼ(xᵢ·xⱼ)−Σ^{l}_{i,=1}αᵢ

Under the conditions Σ^{l}ᵢ₌₁αᵢyᵢ=0 and 0<=αᵢ<=C, i=1, 2 . . . l, wherein C is a given constant.

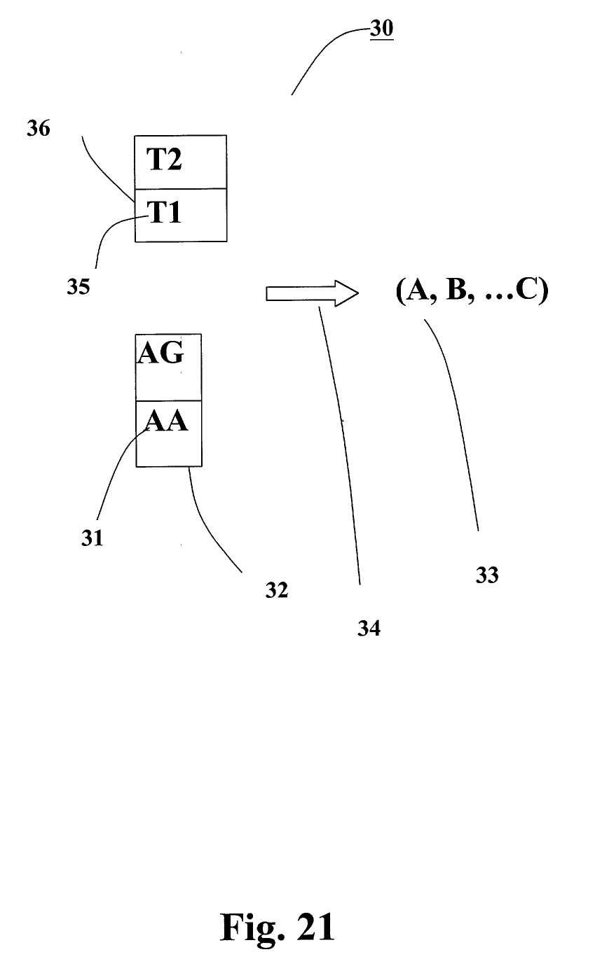

FIG. 21 shows a drawing exemplifying another embodiment according to the present invention. A method 30 comprises the step of representing (arrow 34) a data set, comprising a set of clinical test results T1 and T2 and a set of pairs of genotypes AA and AG, in this example, at SNP locations, as a vector (A,B, . . . C) (reference number 33). The clinical test results, for example, may be the results of a blood test or an MRI. Also, the number and type of clinical test results and number of pairs of genotypes may be varied, as needed.

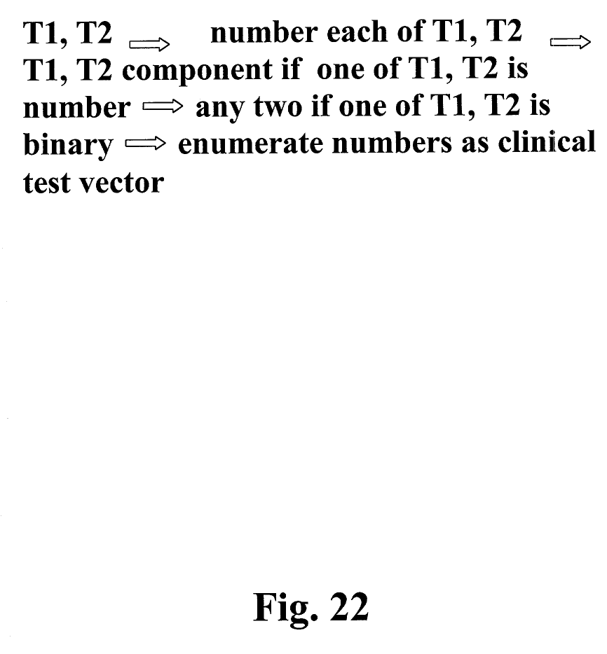

FIG. 22 shows a drawing exemplifying another embodiment according to the present invention, wherein in the method according to FIG. 21, the set of clinical test results T1, T2 is represented as a clinical test vector, according to the following steps: numbering each one of the clinical test results; taking one of the clinical test results as a component of the vector if the one of the clinical test results is a number; choosing any two distinct numbers as a component of the vector if the one of the clinical test results is binary; and enumerating the numbers obtained though above steps as the clinical test vector, in a predetermined order.

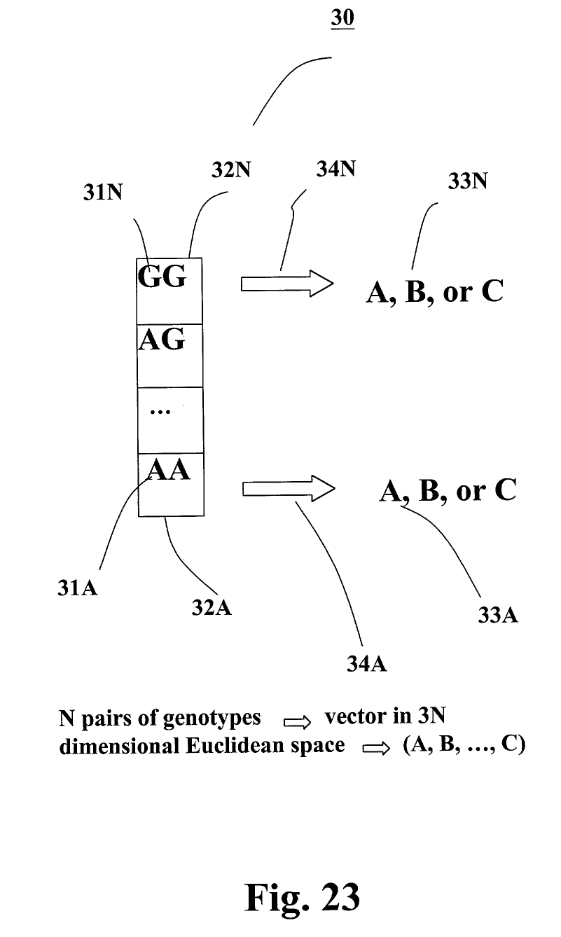

FIG. 23 shows a drawing exemplifying another embodiment according to the present invention, wherein in the method according to FIG. 21, N pairs of genotypes at a respective one of an N number of the plurality of SNP locations are represented as a vector in a 3N dimensional Euclidean space, wherein the vector in a 3N dimensional Euclidean space comprises a N number of the plurality of vectors, in a predetermined order. The order is important and necessary when comparing two different vectors: they need to be in the same order. On the other hand, the particular order may vary as needed so long as the order of vectors that are being compared are the same.

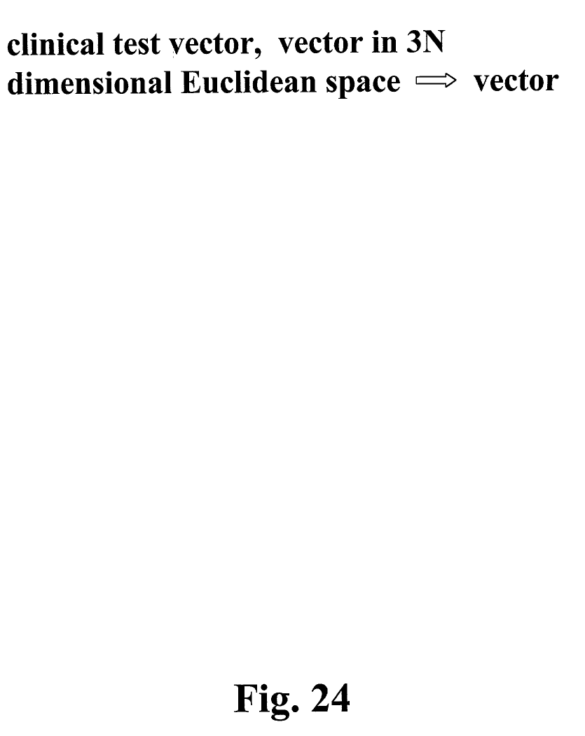

FIG. 24 shows a drawing exemplifying another embodiment according to the present invention, wherein the method according to FIG. 21 further comprises representing the set of clinical test results as a clinical test vector, comprising the following steps: numbering each one of the clinical test results; taking one of the clinical test results as a component of the vector if the one of the clinical test results is a number; choosing any two distinct numbers as a component of the vector if the one of the clinical test results is binary; enumerating the numbers obtained though above steps as the clinical test vector, in a predetermined order; representing N pairs of genotypes at a respective one of an N number of the plurality of SNP locations as a vector in a 3N dimensional Euclidean space, wherein the vector in a 3N dimensional Euclidean space comprises a N number of the plurality of vectors, in a predetermined order; and obtaining a vector comprising the clinical test vector and the vector in a 3N dimensional Euclidean space, in a predetermined order.

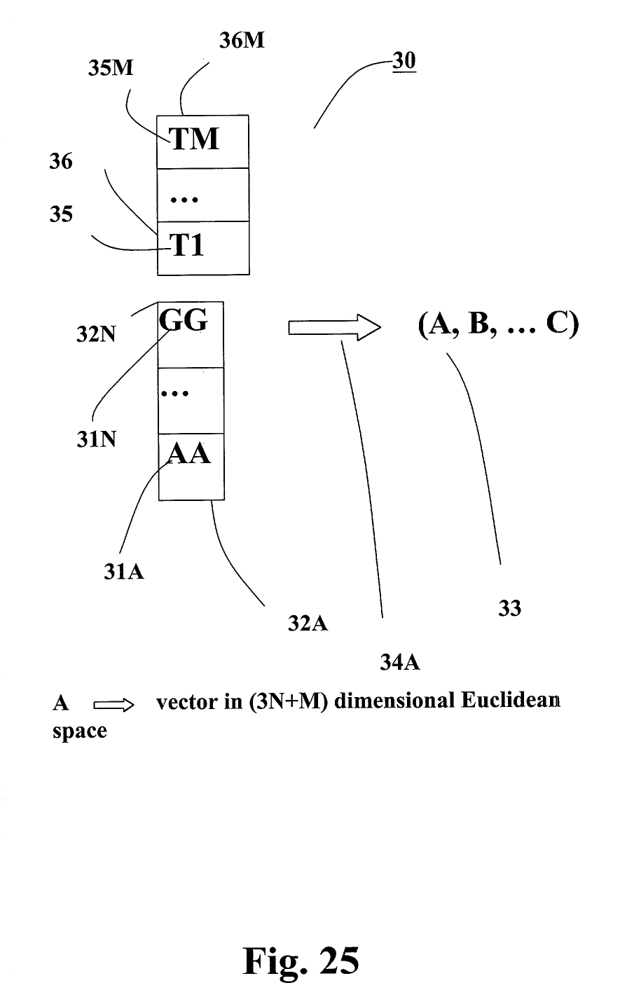

FIG. 25 shows a drawing exemplifying another embodiment according to the present invention, wherein in the method according to FIG. 24, further comprising the following step: representing the data set, comprising a set of clinical test results T1 . . . TM and a set of pairs of genotypes AA . . . GG at a respective one of a plurality of SNP locations, as a vector in a (3N+M)-dimensional Euclidean space, wherein the set of clinical test results comprises M number of test results and the set of pairs of genotypes comprises N pair of genotypes at each respective one of N SNP locations.

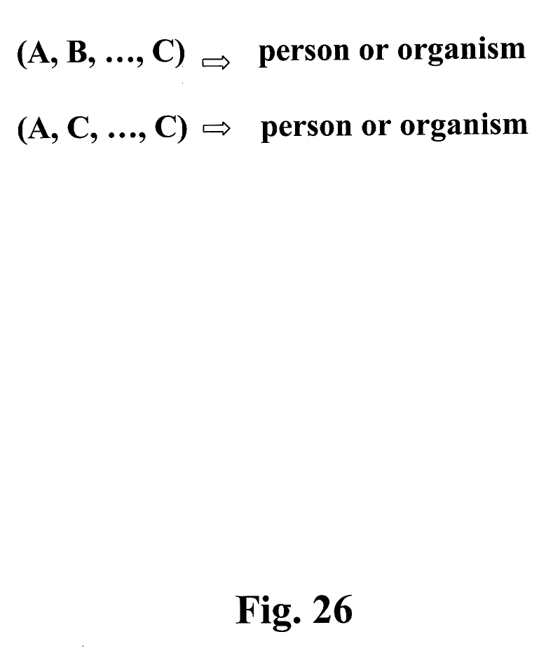

FIG. 26 shows a drawing exemplifying another embodiment according to the present invention, wherein in the method according to FIG. 25, the vector in (3N+M)-dimensional Euclidean space corresponds to a person or an organism, and wherein the person or the organism belongs in one of at least two different classes of a person or an organism, wherein the at least two different classes differ by at least one of a different pair of genotype at an SNP location and a different clinical test result.

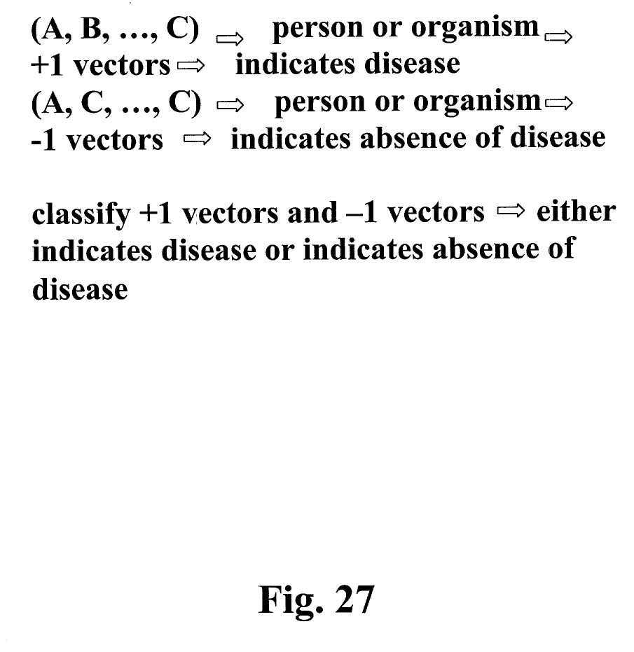

FIG. 27 shows a drawing exemplifying another embodiment according to the present invention, wherein in the method according to FIG. 26, a person or an organism is represented as one of a labeled vector +1 and a labeled vector −1, wherein the labeled vector +1 indicates a disease and the labeled vector −1 indicates absence of the disease. Also, at least two of the labeled vectors corresponding to a respective one of a plurality of the one of a person and an organism are classified into one of at least two subgroups, wherein the first one of the at least two subgroups indicates the disease and the second one of the at least two subgroups indicates absence of the disease.

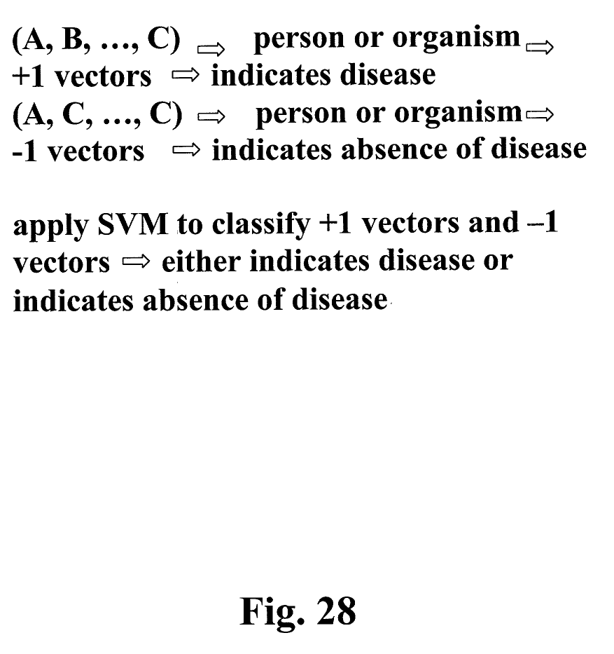

FIG. 28 shows a drawing exemplifying another embodiment according to the present invention, wherein in the method according to FIG. 27, the classifying step further comprises: applying a support vector machine to the at least two labeled vectors so as to optimally classify the at least two labeled vectors into one of the at least two subgroups.

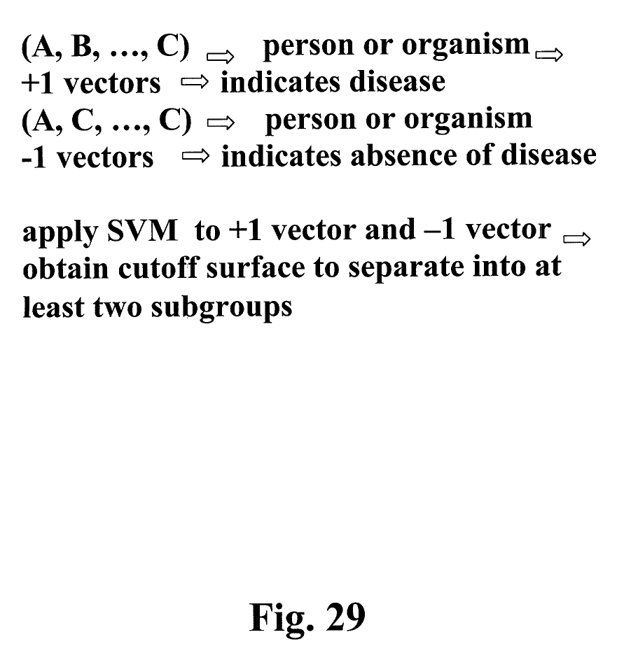

FIG. 29 shows a drawing exemplifying another embodiment according to the present invention, wherein in the method according to FIG. 28, a cutoff hypersurface is obtained by applying the support vector machine to the at least two vectors, wherein the cutoff surface serves to separate and classify the at least two vectors into the at least two subgroups.

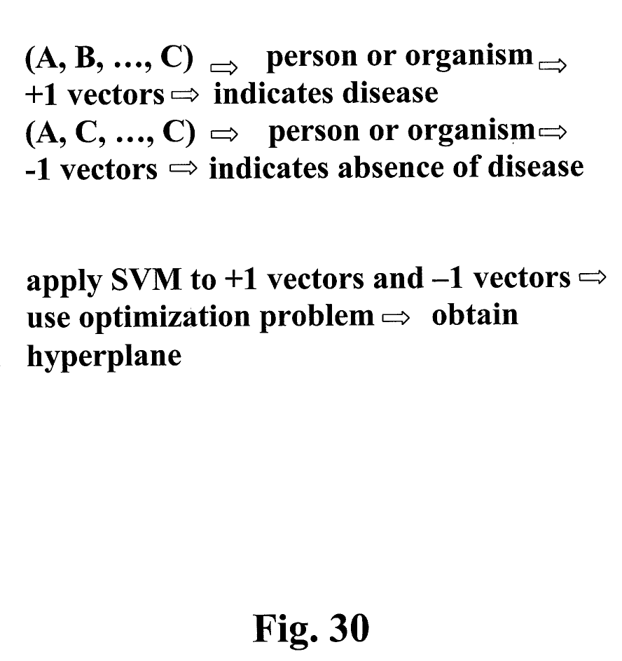

FIG. 30 shows a drawing exemplifying another embodiment according to the present invention, wherein in the method according to FIG. 29, a hyperplane is calculated by using an optimization problem comprising the following, wherein each yᵢ is +1 or −1 and xᵢ is a vector:

Maximize: W(α)=½Σ^{l}_{i,j=1}yᵢyⱼαᵢαⱼ(xᵢ·xⱼ)−Σ^{l}_{i,=1}αᵢ

Under the conditions Σ^{l}ᵢ₌₁αᵢyᵢ=0 and 0<=αᵢ<=C, i=1, 2 . . . l, wherein C is a given constant.

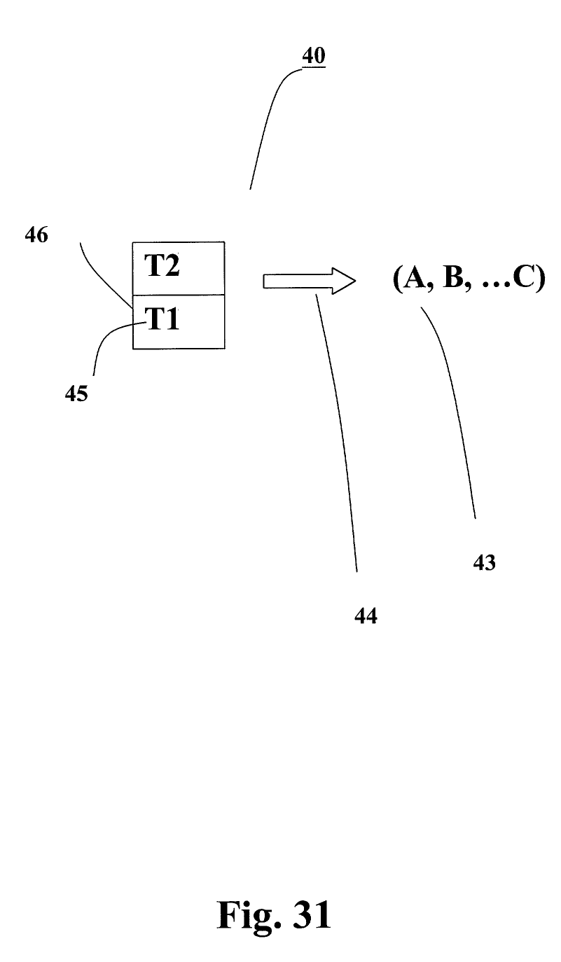

FIG. 31 shows a drawing exemplifying another embodiment according to the present invention. A method 40 comprises the step of representing (arrow 44) a set of clinical test results T1 and T2 as a vector (A,B, . . . C) (reference number 43). Again, the clinical test results, for example, may be the results of a blood test or an MRI. Also, the number and type of clinical test results may be varied, as needed.

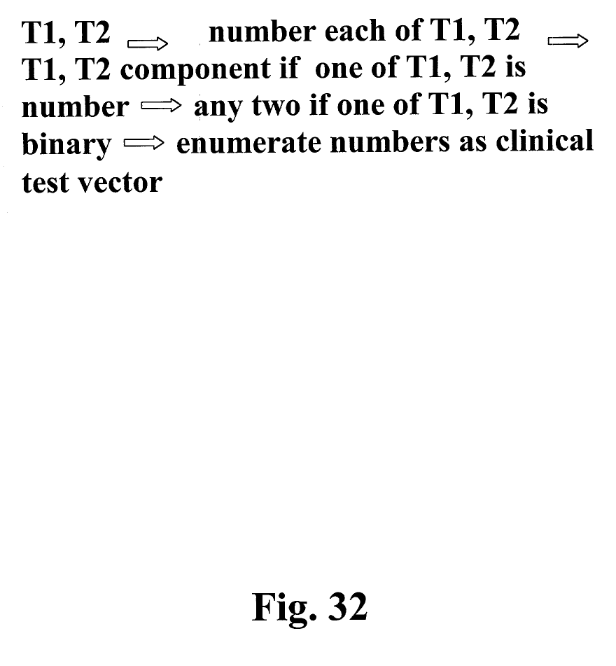

FIG. 32 shows a drawing exemplifying another embodiment according to the present invention, wherein in the method according to FIG. 31, the set of clinical test results T1, T2 is represented as a clinical test vector, according to the following steps: numbering each one of the clinical test results; taking one of the clinical test results as a component of the vector if the one of the clinical test results is a number; choosing any two distinct numbers as a component of the vector if the one of the clinical test results is binary; and enumerating the numbers obtained though above steps as the clinical test vector, in a predetermined order.

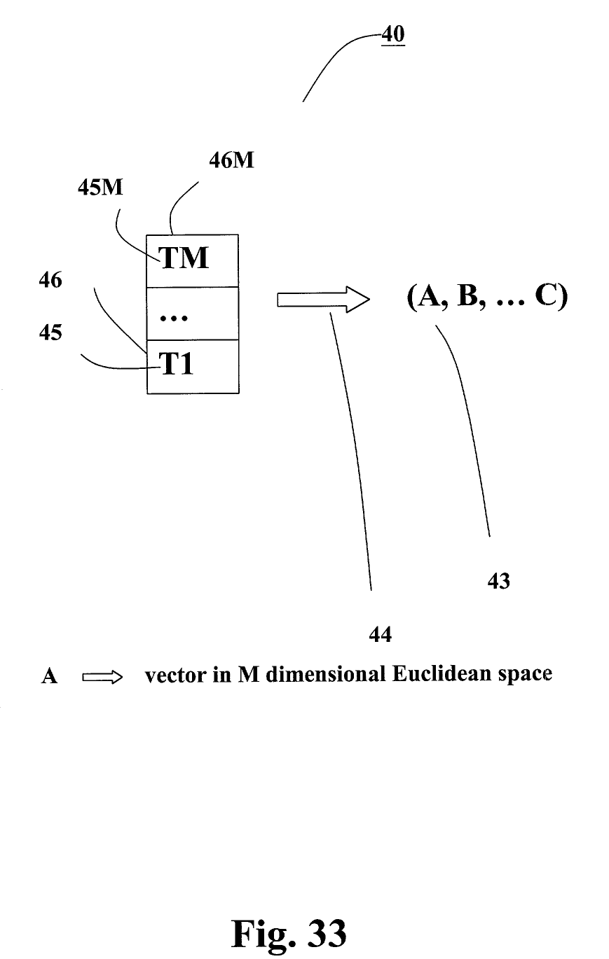

FIG. 33 shows a drawing exemplifying another embodiment according to the present invention, wherein the method according to FIG. 32 further comprises representing the set of clinical test results T1 . . . TM as a vector in a M dimensional Euclidean space, wherein the set of clinical test results comprises M number of test results.

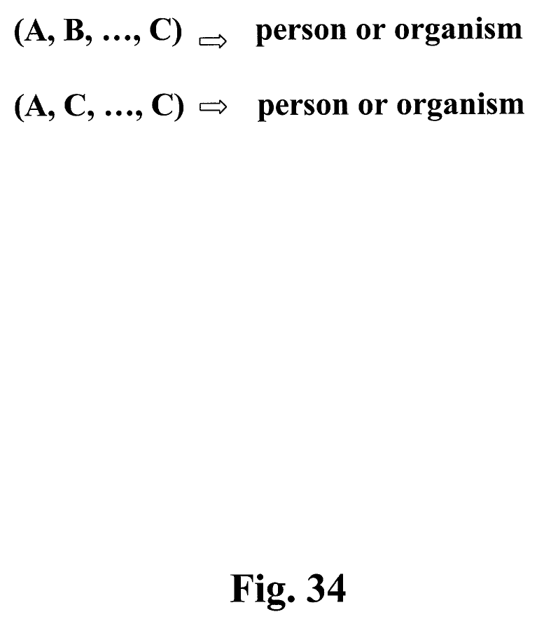

FIG. 34 shows a drawing exemplifying another embodiment according to the present invention, wherein in the method according to FIG. 33, the vector in M dimensional Euclidean space corresponds to a person or an organism, and wherein the person or the organism belongs in one of at least two different classes of a person or an organism, wherein the at least two different classes differ by at least a different clinical test result.

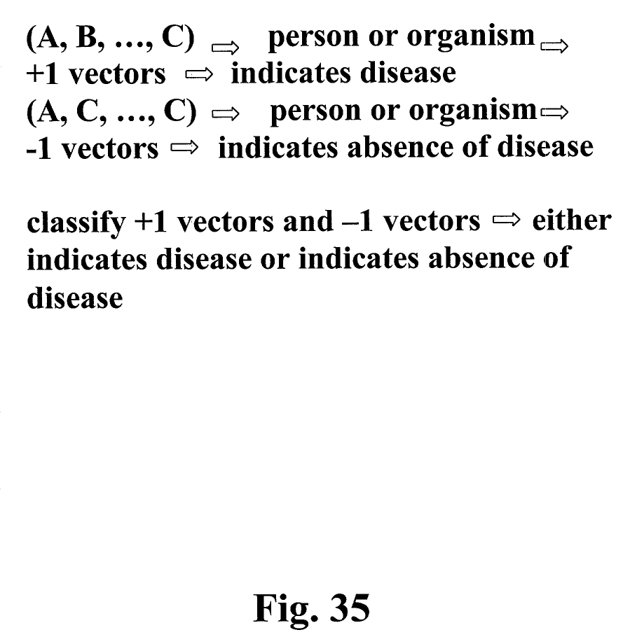

FIG. 35 shows a drawing exemplifying another embodiment according to the present invention, wherein in the method according to FIG. 34, a person or an organism is represented as one of a labeled vector +1 and a labeled vector −1, wherein the labeled vector +1 indicates a disease and the labeled vector −1 indicates absence of the disease. Also, at least two of the labeled vectors corresponding to a respective one of a plurality of the one of a person and an organism are classified into one of at least two subgroups, wherein the first one of the at least two subgroups indicates the disease and the second one of the at least two subgroups indicates absence of the disease.

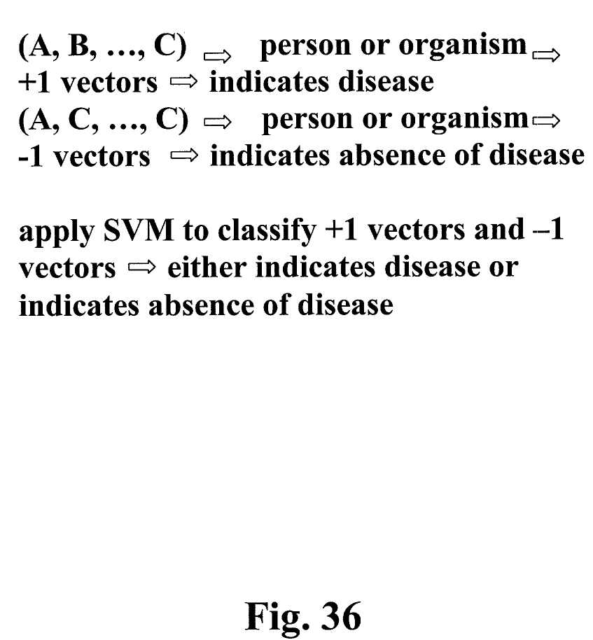

FIG. 36 shows a drawing exemplifying another embodiment according to the present invention, wherein in the method according to FIG. 35, the classifying step further comprises: applying a support vector machine to the at least two labeled vectors so as to optimally classify the at least two labeled vectors into one of the at least two subgroups.

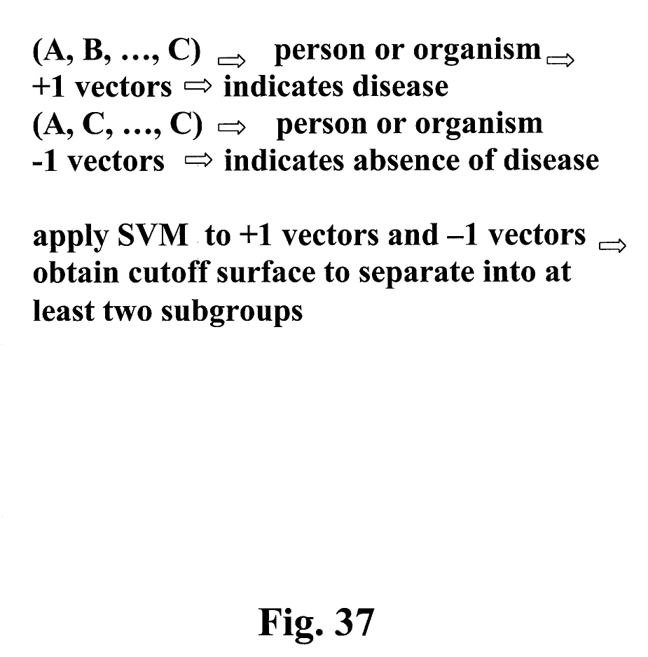

FIG. 37 shows a drawing exemplifying another embodiment according to the present invention, wherein in the method according to FIG. 36, a cutoff hypersurface is obtained by applying the support vector machine to the at least two vectors, wherein the cutoff surface serves to separate and classify the at least two vectors into the at least two subgroups.

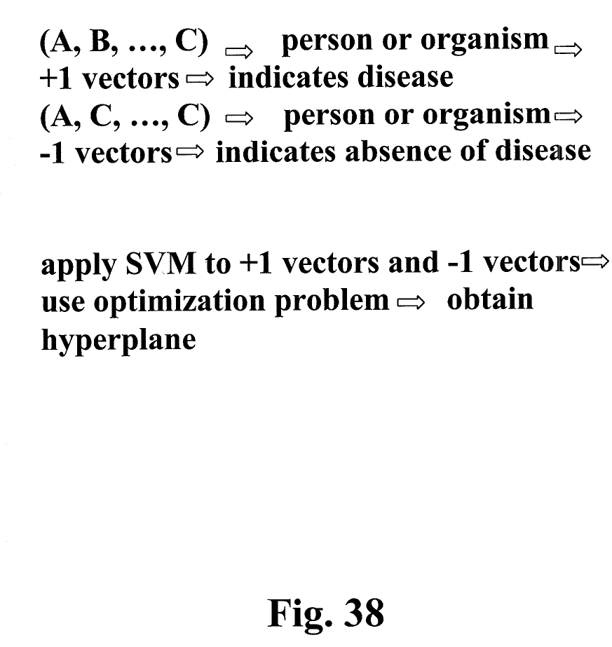

FIG. 38 shows a drawing exemplifying another embodiment according to the present invention, wherein in the method according to FIG. 37, a hyperplane is calculated by using an optimization problem comprising the following, wherein each yᵢ is +1 or −1 and xᵢ is a vector:

Maximize: W(α)=½Σ^{l}_{i,j=1}yᵢyⱼαᵢαⱼ(xᵢ·xⱼ)−Σ^{l}_{i,=1}αᵢ

Under the conditions Σ^{l}ᵢ₌₁αᵢyᵢ=0 and 0<=αᵢ<=C, i=1, 2 . . . l, wherein C is a given constant.

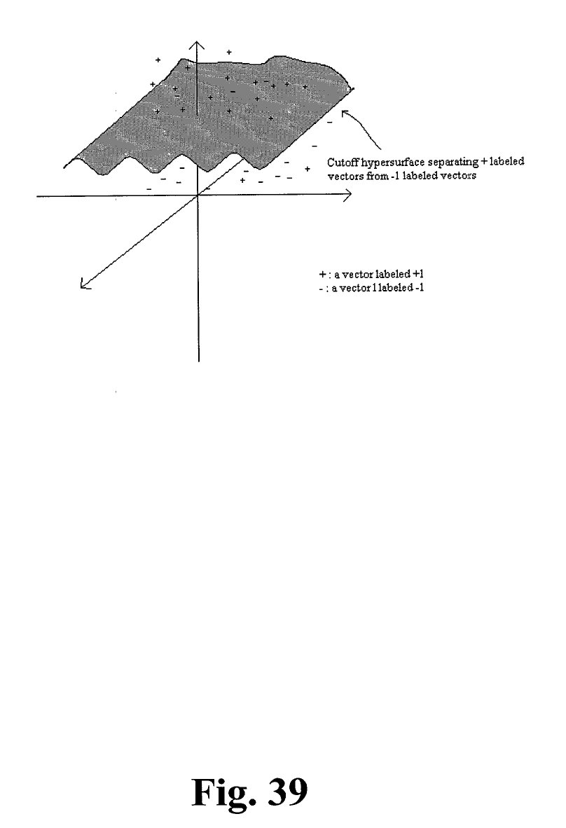

FIG. 39 shows a drawing exemplifying another embodiment according to the present invention, wherein in the cutoff hypersurface as noted above is shown. The shaded hypersurface separates +1 labeled vectors from −1 labeled vectors as indicated.

Although the preferred embodiments of the present invention have been disclosed for illustrative purposes, those skilled in the art will appreciate that various modifications, additions and substitutions are possible, without departing from the scope and spirit of the invention as disclosed in the appended claims.

What is claimed is:

1. A method, comprising the following:
representing a pair of genotypes at an SNP location as a single number.

2. A method according to claim 1, wherein said single number comprises one of A, B, and C, and wherein a relative value of said A,B, and C depend on said SNP location.

3. A method according to claim 2, wherein said A corresponds to a pair of genotypes comprising a wild genotype and a wild genotype, said B corresponds to a pair of genotypes comprising a wild genotype and a mutation genotype, and said C corresponds to a pair of genotypes comprising a mutation genotype and a mutation genotype, and wherein said A, B, and C have distinct values.

4. A method according to claim 1, further comprising the following:
representing each one of a plurality of pairs of genotypes at a respective one of a plurality of SNP locations as a respective one of a plurality of single numbers, wherein said plurality of pairs of genotypes may be represented as a set of single numbers.

5. A method according to claim 4, further comprising the following:
representing N pairs of genotypes at a respective one of an N number of said plurality of SNP locations as a vector in an N dimensional Euclidean space, wherein said vector comprises an N number of said plurality of single numbers, in a predetermined order.

6. A method according to claim 5, wherein said vector corresponds to one of a person and an organism, and wherein said one of a person and an organism belongs in one of at least two different classes of one of a person and an organism, wherein said at least two different classes differ by at least one different pair of genotype at an SNP location.

7. A method according to claim 6, further comprising the following:
representing said one of a person and an organism as one of a labeled vector +1 and a labeled vector −1, wherein said labeled vector +1 indicates a disease and said labeled vector −1 indicates absence of said disease;
classifying at least two of said labeled vectors corresponding to a respective one of a plurality of said one of a person and an organism into either a group with at least two subgroups, wherein the first one of said at least two subgroups indicates the disease and the second one of said at least two subgroups indicates absence of said disease.

8. A method according to claim 7, wherein said classifying step further comprises:
applying a support vector machine to said at least two labeled vectors so as to optimally classify said at least two labeled vectors into one of said at least two subgroups.

9. A method according to claim 8, further comprising the following:
obtaining a cutoff hypersurface by applying said support vector machine to said at least two vectors, wherein said cutoff surface serves to separate and classify said at least two vectors into said at least two subgroups.

10. A method according to claim 9, further comprising the following:
calculating a hyperplane by using an optimization problem comprising the following, wherein each yᵢ is +1 or −1 and xᵢ is a vector:
Maximize: W(α)=½Σ^{l}_{i,j=1}yᵢyⱼαᵢαⱼ(xᵢ·xⱼ)−Σ^{l}_{i,=1}αᵢ
Under the conditions Σ^{l}ᵢ₌₁αᵢyᵢ=0 and 0<=αᵢ<=C, i=1, 2 . . . l, wherein C is a given constant.

11. A method, comprising the following:
representing a pair of genotypes at an SNP location as a vector.

12. A method according to claim 11, wherein said vector comprises one of A, B, and C, and wherein said A, B, and C are vectors that depend on said SNP location.

13. A method according to claim 12, wherein said A corresponds to a pair of genotypes comprising a wild genotype and a wild genotype, said B corresponds to a pair of genotypes comprising a wild genotype and a mutation genotype, and said C corresponds to a pair of genotypes comprising a mutation genotype and a mutation genotype, wherein A, B, and C are three-dimensional vectors, and wherein said A, B, and C have distinct values.

14. A method according to claim 11, further comprising the following:
representing each one of a plurality of pairs of genotypes at a respective one of a plurality of SNP locations as a respective one of a plurality of vectors, wherein said plurality of pairs of genotypes may be represented as a vector comprising said plurality of vectors.

15. A method according to claim 14, further comprising the following:
representing N pairs of genotypes at a respective one of an N number of said plurality of SNP locations as a vector in a 3N dimensional Euclidean space, wherein said vector in a 3N dimensional Euclidean space comprises a N number of said plurality of vectors, in a predetermined order.

16. A method according to claim 15, wherein said vector in 3N dimensional Euclidean space corresponds to one of a person and an organism, and wherein said one of a person and an organism belongs in one of at least two different classes of one of a person and an organism, wherein said at least two different classes differ by at least one different pair of genotype at an SNP location.

17. A method according to claim 16, further comprising the following:
representing said one of a person and an organism as one of a labeled vector +1 and a labeled vector −1, wherein said labeled vector +1 indicates a disease and said labeled vector −1 indicates absence of said disease;
classifying at least two of said labeled vectors corresponding to a respective one of a plurality of said one of a person and an organism into one of at least two subgroups, wherein the first one of said at least two subgroups indicates the disease and the second one of said at least two subgroups indicates absence of said disease.

18. A method according to claim 17, wherein said classifying step further comprises:
applying a support vector machine to said at least two labeled vectors so as to optimally classify said at least two labeled vectors into one of said at least two subgroups.

19. A method according to claim 18, further comprising the following:
obtaining a cutoff hypersurface by applying said support vector machine to said at least two vectors, wherein said cutoff surface serves to separate and classify said at least two vectors into said at least two subgroups.

20. A method according to claim 19, further comprising the following:
calculating a hyperplane by using an optimization problem comprising the following, wherein each yᵢ is +1 or −1 and xᵢ is a vector:
Maximize: W(α)=½Σ^{l}_{i,j=1}yᵢyⱼαᵢαⱼ(xᵢ·xⱼ)−Σ^{l}_{i,=1}αᵢ
Under the conditions Σ^{l}ᵢ₌₁αᵢyᵢ=0 and 0<=αᵢ<=C, i=1, 2 . . . l, wherein C is a given constant.

21. A method, comprising the following:
representing a data set, comprising a set of clinical test results and a set of pairs of genotypes at a respective one of a plurality of SNP locations, as a vector.

22. A method according to claim 21, further comprising the following:
representing said set of clinical test results as a clinical test vector, comprising the following:
numbering each one of said clinical test results;
taking one of said clinical test results as a component of said vector if said one of said clinical test results is a number;
choosing any two distinct numbers as a component of said vector if said one of said clinical test results is binary; and
enumerating said numbers obtained though above steps as said clinical test vector, in a predetermined order.

23. A method according to claim 21, further comprising the following:
representing N pairs of genotypes at a respective one of an N number of said plurality of SNP locations as a vector in a 3N dimensional Euclidean space, wherein said vector in a 3N dimensional Euclidean space comprises a N number of said plurality of vectors, in a predetermined order.

24. A method according to claim 21, further comprising the following:
representing said set of clinical test results as a clinical test vector, comprising the following:
numbering each one of said clinical test results;
taking one of said clinical test results as a component of said vector if said one of said clinical test results is a number;
choosing any two distinct numbers as a component of said vector if said one of said clinical test results is binary;
enumerating said numbers obtained though above steps as said clinical test vector, in a predetermined order;
representing N pairs of genotypes at a respective one of an N number of said plurality of SNP locations as a vector in a 3N dimensional Euclidean space, wherein said vector in a 3N dimensional Euclidean space comprises a N number of said plurality of vectors, in a predetermined order; and
obtaining a vector comprising said clinical test vector and said vector in a 3N dimensional Euclidean space, in a predetermined order.

25. A method according to claim 24, further comprising the following:
representing said data set, comprising a set of clinical test results and a set of pairs of genotypes at a respective one of a plurality of SNP locations, as a vector in a (3N+M)-dimensional Euclidean space, wherein said set of clinical test results comprises M number of test results and said set of pairs of genotypes comprises N pair of genotypes at each respective one of N SNP locations.

26. A method according to claim 25, wherein said vector in (3N+M)-dimensional Euclidean space corresponds to one of a person and an organism, and wherein said one of a person and an organism belongs in one of at least two different classes of one of a person and an organism, wherein said at least two different classes differ by at least one of a different pair of genotype at an SNP location and a different clinical test result.

27. A method according to claim 26, further comprising the following:
representing said one of a person and an organism as one of a labeled vector +1 and a labeled vector −1, wherein said labeled vector +1 indicates a disease and said labeled vector −1 indicates absence of said disease;
classifying at least two of said labeled vectors corresponding to a respective one of a plurality of said one of a person and an organism into one of at least two subgroups, wherein the first one of said at least two subgroups indicates the disease and the second one of said at least two subgroups indicates absence of said disease.

28. A method according to claim 27, wherein said classifying step further comprises:
applying a support vector machine to said at least two labeled vectors so as to optimally classify said at least two labeled vectors into one of said at least two subgroups.

29. A method according to claim 28, further comprising the following:
obtaining a cutoff hypersurface by applying said support vector machine to said at least two vectors, wherein said cutoff surface serves to separate and classify said at least two vectors into said at least two subgroups.

30. A method according to claim 29, further comprising the following:
calculating a hyperplane by using an optimization problem comprising the following, wherein each yᵢ is +1 or −1 and xᵢ is a vector:
Maximize: W(α)=½Σ^{l}_{i,j=1}yᵢyⱼαᵢαⱼ(xᵢ·xⱼ)−Σ^{l}_{i,=1}αᵢ
Under the conditions Σ^{l}ᵢ₌₁αᵢyᵢ=0 and 0<=αᵢ<=C, i=1, 2. . . l, wherein C is a given constant.

31. A method, comprising the following:
representing a set of clinical test results as a vector.

32. A method according to claim 31, wherein said representing step comprising the following:
numbering each one of said clinical test results;
taking one of said clinical test results as a component of said vector if said one of said clinical test results is a number;
choosing any two distinct numbers as a component of said vector if said one of said clinical test results is binary; and
enumerating said numbers obtained though above steps as said clinical test vector, in a predetermined order.

33. A method according to claim 32, further comprising the following:
representing said set of clinical test results as a vector in an M dimensional Euclidean space, wherein said set of clinical test results comprises M number of test results.

34. A method according to claim 33, wherein said vector in M dimensional Euclidean space corresponds to one of a person and an organism, and wherein said one of a person and an organism belongs in one of at least two different classes of one of a person and an organism, wherein said at least two different classes differ by at least a different clinical test result.

35. A method according to claim 34, further comprising the following:
representing said one of a person and an organism as one of a labeled vector +1 and a labeled vector −1, wherein said labeled vector +1 indicates a disease and said labeled vector −1 indicates absence of said disease;
classifying at least two of said labeled vectors corresponding to a respective one of a plurality of said one of a person and an organism into one of at least two subgroups, wherein the first one of said at least two subgroups indicates the disease and the second one of said at least two subgroups indicates absence of said disease.

36. A method according to claim 35, wherein said classifying step further comprises:
applying a support vector machine to said at least two labeled vectors so as to optimally classify said at least two labeled vectors into one of said at least two subgroups.

37. A method according to claim 36, further comprising the following:
obtaining a cutoff hypersurface by applying said support vector machine to said at least two vectors, wherein said cutoff surface serves to separate and classify said at least two vectors into said at least two subgroups.

38. A method according to claim 37, further comprising the following:
calculating a hyperplane by using an optimization problem comprising the following, wherein each y(i) is +1 or −1 and x(i) is a vector:
Maximize: W(α)=½Σ^{l}_{i,j=1}yᵢyⱼαᵢαⱼ(xᵢ·xⱼ)−Σ^{l}_{i,=1}αᵢ
Under the conditions Σ^{l}ᵢ₌₁αᵢyᵢ=0 and 0<=αᵢ<=C, i=1, 2 . . . l, wherein C is a given constant.
